(19) United States
(12) Patent Application Publication
(10) Pub. No.: US 2018/0137933 A1
(43) Pub. Date: May 17, 2018

(54) Computer-Implemented System and Method for Recruiting Patients for Quadruple-Blind Clinical Studies

(71) Applicant: Michael Burgio, Palm Bay, FL (US)

(72) Inventor: Michael Burgio, Palm Bay, FL (US)

(21) Appl. No.: 15/870,656

(22) Filed: Jan. 12, 2018

Related U.S. Application Data
(63) Continuation of application No. 14/680,373, filed on Apr. 7, 2015.
Application No. 14/680,373 is a continuation of application No. 14/680,389, filed on Apr. 7, 2015.

(51) Int. Cl. G16H 10/60; G16H 10/20; G16H 40/20; G06Q 50/18

(52) U.S. Cl. CPC G16H 10/60; G06Q 50/18; G16H 40/20; G16H 10/20

(57) ABSTRACT

A system and method for enrolling patients in a medical study, includes: a database component operative to maintain a medical practice database component and their corresponding plurality of specialties and a quadruple-blind clinical studies database component and their corresponding plurality of medical studies. The system further includes a communications component to alert the medical practices of the medical studies and observe changes to the database components. The system also includes a processor programmed to update the database components, periodically match compatible medical specialties with the medical studies, generate reports of the matched medical practices in the medical practice database, and generate reports to the FDA. The system also includes a compensation database component utilizing a processor to determine compensation for participants in the conducting the study.

### CROSS-REFERENCE TO RELATED APPLICATIONS

This application is a continuation application of U.S. Non-Provisional Utility application Ser. No. 14/680,373 filed on Apr. 7, 2015. This application is also is a continuation application of U.S. Non-Provisional Utility application Ser. No. 14/680,389 filed on Apr. 7, 2015. The contents of both application is incorporated by this reference.

### FIELD OF THE DISCLOSURE

The present invention is in the field of clinical study participant recruitment, and, more specifically, in the field of clinical study participant recruitment for quadruple-blind clinical studies.

### BACKGROUND

Many companies sponsor clinical studies for new drugs, medical devices, therapies, or treatment programs. Typical clinical study sponsors include pharmaceutical companies, biotech companies, medical device companies, clinical research organizations (CRO's), and site management organizations (SMO's). Clinical studies are often an important step before obtaining FDA approval for particular drugs. As used herein, the term “clinical study” is used interchangeably with the terms “clinical trial,” “research study,” “medical study,” and “medical research study.” Moreover, the term “medical researcher” will be used to collectively refer to “research physicians,” “researchers,” or the like.

Patients who have been diagnosed with a disease are often in need of finding appropriate clinical studies for new drugs, medical devices, or treatments to treat their disease. Patients with serious diseases may only have weeks or months to live, and thus the ability to find available clinical studies and information about those studies quickly and efficiently is invaluable.

Likewise, the demand for clinical study participants, however, typically is greater than the supply of clinical study participants. Many factors contribute to this imbalance, including the difficulty of identifying and recruiting potential participants and, in many countries, the statutory proscription against offering a significant financial incentive to potential participants once they have been identified.

Additionally, as the number of elderly people increase in the United States and their lifespans extend there is an ever increasing need for newer and safer pharmaceutical products. As such, there is a need for new drugs and medical devices to be approved more rapidly. With the mapping of the human genome it is estimated that drug targets and drugs will multiply tenfold, necessitating more clinical testing. In fact, The

Pharmaceutical Research and Manufacturers of America (PhRMA) states that all drugs currently on the market are based on about 500 different targets. They expect this number to increase 600-2000%, to 3,000 to 10,000 drug targets in the coming years. However, such medical advances are outrageously expensive and have necessitated changes throughout the industry.

It is estimated to cost $880 million to bring one new drug to market. And it is estimated that the average pharmaceutical company has 70 new drugs in development. This has forced the pharmaceutical companies to consolidate for the purpose of underwriting the prohibitive expense of bringing a drug to market. The average drug takes 10 to 12 years to bring to market and must negotiate a series of 3 clinical trials before approval by the Food and Drug Administration (FDA) can even be granted, leaving 8 to 10 years on a drug patent to recoup costs and turn a profit. Factoring in the governmental and managed care cost containment pressures, the pharmaceutical companies must produce one successful medicine or medical device every 18 months to survive.

Going forward, the pharmaceutical companies are in a position where they are producing more new drug compounds than ever before because they may lose the patents on many of their highly profitable drugs. They are also being squeezed by the managed care industry. It is therefore critical for the pharmaceutical companies to discover, test and market the maximum number of new drugs in the minimum amount of time. Accordingly, the present invention addresses the foregoing shortcomings.

In addition, clinical studies come in various forms. Traditionally, clinical studies utilized a double-blind study. Double-blind describes an especially stringent way of conducting an experiment which attempts to eliminate subjective, unrecognized biases carried by an experiment's patients (usually human) and conductors. In most cases, double-blind clinical studies are held to achieve a higher standard of scientific rigor than blind or non-blind clinical studies. In these double-blind clinical studies, neither the participants nor the researchers know which participants belong to the control group, nor the test group. Only after all data have been recorded (and in some cases, analyzed) do the researchers learn which participants were exposed to a placebo or an established drug/device. In this way, misconceptions or prejudices are supposedly eliminated.

Unfortunately, there are significant problems with double-blind clinical studies. For example, the use of alcohol, cigarettes, and caffeine, as well as nutritional supplements are typically ignored in double-blind clinical studies unless said study specifically involves smokers or alcohol users as key variables or confounding factors. Yet, these may have major impacts on the results of other studies, particularly those on subtle brain functions such as depression and anxiety. In the same way, double-blind clinical studies are often too short term. A classic example is from psychiatry: Most antidepressant studies are only six to eight weeks long, looking at acute management of depression. However, most double-blind studies do not evaluate long-term efficacy of drugs. In light of this, the quadruple-blind clinical study methodology was designed wherein the subjects, investigator(s), evaluator, and the data analysts remain blinded. The mathematical difference between a double-blind clinical study and a quadruple-blind clinical study, is a 400% increase in the efficacy, accuracy of certain clinical findings, according to one study. This is because a quadruple-blind clinical study conceals information about the clinical study that might lead to bias; reduces the observer influence from experimenters, research physicians, and the like; and reduces conscious deception enabling a more objective assessment with regards to the clinical study. This increase in efficacy provides much more accurate information to the FDA, government entities, the sponsors funding these studies, and most importantly, the patients.

Regrettably, today there is no efficient system of locating suitable patients for quadruple-blind clinical studies, despite the proven efficacy of the quadruple-blinded methodology. As a result, the present invention solves the aforementioned problem where patients are denied access to quadruple-blind clinical studies, which could lead to a faster diagnosis of a condition, and in turn a faster treatment, faster recovery time, and better quality of life. Because there is no effective system for quickly matching qualified patients with quadruple-blind clinical studies, doctors are often not aware of all the quadruple-blind clinical studies that are being performed in different geographic regions, and they lack the wherewithal to locate qualified patents. In addition, quadruple-blind clinical study sponsors have difficulty finding suitable patients for their studies because there is a lack of up-to-date listings of quadruple-blind clinical studies, patients are geographically dispersed, many quadruple-blind clinical studies require screening large segments of the population, and patients lack insurance coverage. Additionally, patients suffer from consumer confusion with regard to medical terminology and protocol information, and thus have a difficult time identifying appropriate quadruple-blind clinical studies.

Quadruple-blind clinical study sponsors are also hurt by this problem, since the inability to quickly find acceptable patients to enroll in their quadruple-blind clinical studies delays development of their new drugs or devices and thereby delays FDA approval in certain circumstances. In light of the foregoing, what is needed is a system and method of efficiently matching qualified patients with suitable quadruple-blind clinical studies. What is also needed is a system and method that can match patient medical profiles and patient characteristics with quadruple-blind clinical study acceptance criteria for a wide range of quadruple-blind clinical studies in dispersed geographic areas. Lastly, there exists a need to provide a global means to facilitate the identification and communication between clinical study sponsors, medical practices, medical researchers, and eligible clinical patients to expedite the process of launching quadruple-blind clinical studies in an efficient and cost-effective manner.

### SUMMARY

The present disclosure addresses the above stated situation by providing, in an embodiment, a computer-implemented system and method for recruiting patients for quadruple-blind clinical studies, comprising: a database component operative to maintain and to store a medical practice database component having a plurality of medical practices each identified by a corresponding specialty, a quadruple-blind clinical studies database component having a plurality of quadruple-blind clinical studies, a protocol database component having a plurality of quadruple-blind clinical study protocols, a patient database component having a plurality of patient records, each of said plurality of patient records identifying a patient and the patient's association with at least one of said plurality of medical practices in said medical practice database component, a unique identifiers database component having a plurality of unique identifiers, a BES database component including a plurality of contract research organizations, a verification database component coupled to the communications component and further adapted to verify a medical researchers compliance with state and federal licensing requirements, a compensation database component having a plurality of decision rules, wherein said decision rules are implemented to compensate a participant in said quadruple-blind clinical study based on a binding legal written instrument, wherein said binding written legal agreement complies with 42 CFR 1001.952(d); a communications component, coupled to the plurality of clinical study entities and to the plurality of medical practices through a communications link, to electronically said medical practices in real time to the existence of said clinical studies, to electronically notify said clinical study entities to the existence of said medical practices in real time, to electronically notify said clinical study entities and said medical practices to the existence of patients suitable for enrollment in a quadruple-blind clinical study based upon information from said database components and to receive changes to said database components, to receive by a computer device a plurality of input data from said medical practices via an interactive graphical user interface comprising fields of required data pre-defined by the computer device; a registration component configured to communicate with said database components utilizing said communications component to present one or more web pages that allow a user to register with said patient database component, by submitting registration information to said patient database component, wherein the registration information includes at least a geographic location of the user, at least one disease condition of interest to the user, contact information, and permission information indicating whether the user wishes to receive electronic notice of said plurality of quadruple-blind clinical studies; a processor, coupled to said communications component and to said database components, configured to update said database components, to match at least one of said plurality of medical practices with at least one of said plurality of quadruple-blind clinical studies, to query said patient database component to identify data that indicate patients who may be suitable for said quadruple-blind clinical studies, wherein said query includes searching for key words embedded in the patient database records, to match patients suitable for enrollment with said medical studies based upon information from said database components, to generate a report of medical practices matched to said clinical studies, to generate a report of patients matched to said quadruple-blind clinical studies, to utilize said communications component so as to electronically notify in real time said medical practices associated with said matched patients, and to calculate, utilizing said compensation database component, a fee to be paid to a participant for referral procedures performed in referral of said matched patients to said quadruple-blind clinical study to which said patient is matched; and wherein said communications component sends at least one of said reports over said communications link to said medical practices in said medical practice database to electronically notify in real time said medical practices of said matched patients for referral, and wherein said medical practices locate and refer said matched patients to said quadruple-blind clinical study to which each said patient is matched; and wherein said medical practices electronically notify in real time said clinical study entities of referral of said patients to said medical studies over said communications link utilizing said communication component, and said database components are updated utilizing said processor.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a schematic diagram of the system according to the present invention;

FIG. 2 depicts an exemplary contract, or consultation agreement, illustrating a binding legal written instrument wherein a medical researcher is compensated by a medical practice for participating in a quadruple-blind clinical study, in accordance with one embodiment of the present invention;

FIG. 3 depicts an exemplary contract, or license agreement, illustrating a binding legal written instrument wherein a sponsor contracts with a BES to administer a quadruple-blind clinical study, in accordance with one embodiment of the present invention;

FIG. 4. depicts an exemplary Physician Compensation Report compiled by Medscape to be utilized in determining the hourly rate of a medical researcher based on their corresponding area of medicine with respect to their participation in a quadruple-blind medical study, in accordance with one embodiment of the present invention; and,

FIG. 5 is a flowchart of the method according to the present invention.

### DETAILED DESCRIPTION

Although the following discloses example systems and methods including, among other components, software executed on hardware, it should be noted that such methods and apparatus(es) are merely illustrative and should not be considered as limiting. For example, it is contemplated that any or all of these hardware and software components could be embodied exclusively in hardware, exclusively in software, exclusively in firmware, or in any combination of hardware, software, and/or firmware. Accordingly, while the following describes example systems and methods, the examples provided are not the only way to implement such methods and systems.

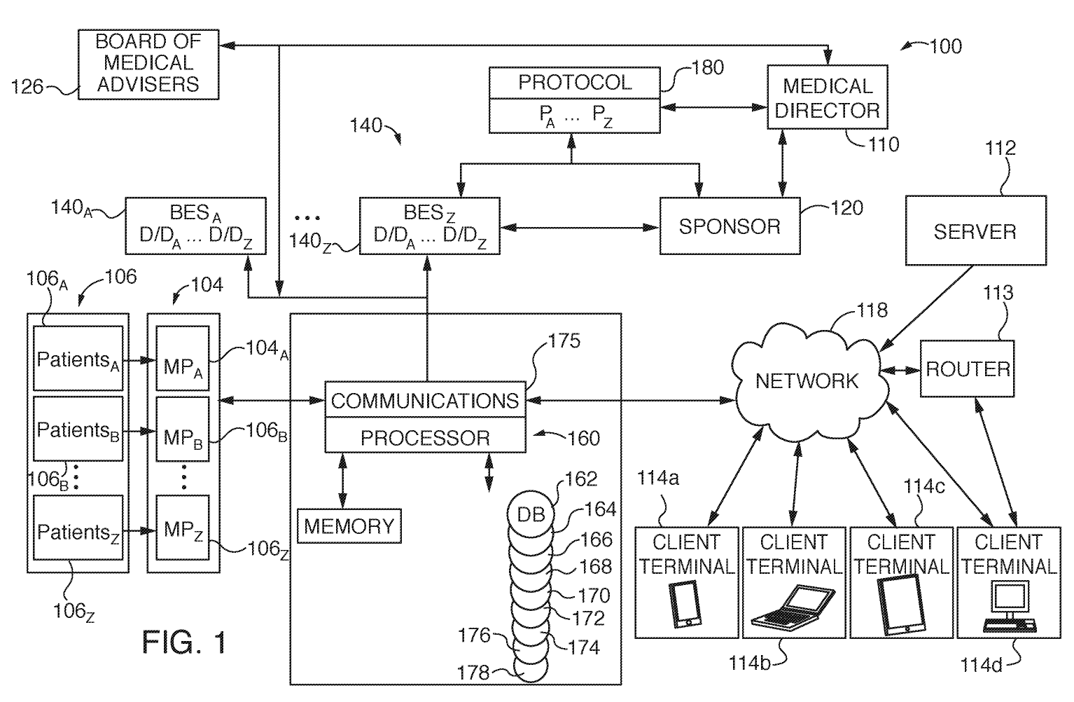

As used herein, the term tangible computer-readable medium is expressly defined to include any type of computer-readable medium and to expressly exclude propagating signals. Example computer-readable medium include, but are not limited to, a volatile and/or non-volatile memory, a volatile and/or non-volatile memory device, a compact disc (CD), a digital versatile disc (DVD), a floppy disk, a read-only memory (ROM), a random-access memory (RAM), a programmable ROM (PROM), an electronically-programmable ROM (EPROM), an electronically-erasable PROM (EEPROM), an optical storage disk, an optical storage device, magnetic storage disk, a magnetic storage device, a cache, and/or any other storage media in which information is stored for any duration (e.g., for extended time periods, permanently, brief instances, for temporarily buffering, and/or for caching of the information) and which can be accessed by a processor, a computer and/or other machine having a processor, such as the example processor discussed below in connection with FIG. 1. As used herein, the term non-transitory computer-readable medium is expressly defined to include any type of computer-readable medium.

Referring now to FIG. 1, it can be seen that a system and related method for recruiting and enrolling patients in a quadruple-blind clinical study from a plurality of medical practices is designated generally by the numeral 100. The system 100, which may be implemented within the United States or internationally, may include a server 112, a communications network 118, and one or more participant terminals 114a-114d (collectively 114). The different elements and components of the system 100 may communicate with one another using wireless communications or hardwired connections, such as fiber optics, cable, DSL, telephone lines, and other similar connections.

The communications network 118 may include any number of networks capable of providing communications between the server 112, system 100, client terminals 114, sponsors 120, medical director 110, board of medical advisers 126, protocols 180, BES 140, medical practices 104, and patients 106. For example, the communications network may be one or more, or any combination of, wireless networks, data or packet networks, publicly switched telephone networks (PSTN), etc.

The client terminals 114 may include any suitable device operable to act as a client on a network. Illustrative terminals 114 include, but are not limited to, personal computers, desktop computers, laptop computers, servers, or any suitable telecommunications device, including, but not limited to, VoIP telephones, smart telephones or wireless devices, such as cellular telephones, personal digital assistants (PDA), communications enabled mp3 players, etc. Each terminal 114 may be configured to transmit and/or receive information to and/or from both the server 112 and the system 100.

The system 100, includes various organizations or entities that cooperate with one another to enroll patients in a quadruple-blind clinical study, provide results of the study, and distribute payments to various participants of the quadruple-blind clinical study. In particular, the system 100 includes a pharmaceutical or medical device company, hereinafter collectively referred to as “sponsors” or “sponsor” designated generally by the numeral 120. It is to be appreciated that the sponsors 120 may be located internationally. As discussed previously, sponsors are required to assure the consuming public that the medical drugs and/or devices that they manufacture are safe for use and/or consumption and that they have no adverse consequences resulting from their use. In order to obtain an independent evaluation of the drug or medical device, the sponsors 120 contract with an entity identified by the initialism BES designated generally by the numeral 140. It will be appreciated that sponsors may contract a study for a particular drug or device with various BESs and as such they are designated with an appropriate alphabetic suffix. A BES 140 may be an entity such as a Contract Research Organization which procures drug trial contracts with global reach so that they can either perform the trials themselves, or outsource it; a Site Management Organization which manages multiple research sites, inasmuch as these organizations do research at multiple places or facilities; or a Researcher which may be an individual doctor or medical professional, either individually or in a group, that does research as a part of their professional medical services. Accordingly, a BES may be any entity with its state of incorporation or principle place of business within the United States, alternatively, or with its principle place of business based internationally, that administers clinical studies and hence has a need to recruit patients for these studies.

Another set of participants in the system 100 are medical practices designated generally by the numeral 104 wherein any number of specific medical practices are provided with an alphabetic suffix. Accordingly, the system 100 is adaptable to communicate with various types of medical practices. As used herein, the terms “clinic(s)” and “medical practice(s)” are interchangeable and synonymous. For example, medical practice 104A may be a medical practice specializing in chiropractic services, while medical practice 104B may be a medical practice specializing in cardiology. Likewise, medical practice 104Z may be a hospital practice that specializes in emergency medicine. Each medical practice 104 has associated therewith a patient clientele designated as 106 with a corresponding alphabetic suffix associated with the alphabetic suffixes of the medical practice 104.

A BES 140 may be requested to evaluate a drug or device, which is designated in the drawings as D/D, wherein different drugs or devices are provided with a different alphabetic suffix as deemed appropriate by the BES 140. The BES 140, in conjunction with the sponsors 120, prepare a protocol 180 for a quadruple-blind clinical study to be stored in the system 100, which is designated in the drawings as an alphabetic suffix. As will be discussed in further detail, the protocol 180 is a document that describes how a clinical trial will be conducted (the objective(s), design, methodology, statistical considerations and organization of a clinical study,) and ensures the safety of the patients and integrity of the data collected, which are submitted to a medical director designated generally by the numeral 110. If desired, the sponsor 120 may directly establish a relationship with the medical director 110 for the purpose of obtaining prospective patients for a clinical study.

The medical director 110 is an entity that assists the BESs 140 in finding patients that meet the eligibility requirements established by the sponsors 120 for the respective drug or device. The medical director 110, BES 140, board of medical advisers 126, sponsor 120 are capable of receiving and sending communications in any number on forms through the system 100, including, but not limited to email, personal computers, desktop computers, laptop computers, servers, or any suitable telecommunications device, including, but not limited to, VoIP telephones, smart telephones or wireless devices, such as cellular telephones, personal digital assistants (PDA), communications enabled mp3 players, client terminals 114, etc.

The system 100 includes a computer processor 160 which includes the necessary hardware, software and tangible computer-readable medium (hereinafter “memory”) to implement the system and methodologies disclosed herein. However, it will be appreciated that the system 100 may be implemented in hardware, software, or a suitable combination of hardware and software, and which can be one or more software systems operating on a general purpose server platform. As used herein, a hardware system can include discrete semiconductor devices, an application-specific integrated circuit, a field programmable gate array or other suitable devices. A software system can include one or more objects, agents, threads, lines of code, subroutines, separate software applications, user-readable (source) code, machine-readable (object) code, two or more lines of code in two or more corresponding software applications, databases, or other suitable software architectures. In one exemplary embodiment, a software system can include one or more lines of code in a general purpose software application, such as an operating system, and one or more lines of code in a specific purpose software application. Alternatively, the functions of the server may be performed by a human user.

The processor 160 is programmed to coordinate all activities of the system such as routing electronic communications, performing searches of the database 162, generating compensation statements for various participants of the quadruple-blind clinical studies, generating reports pertaining to the requirements and results of the quadruple-blind clinical studies, and facilitating the quadruple-blind clinical study process. As indicated above, the processor 160 provides access to a database designated generally by the numeral 162. The processor 160 also formats the contents of the database and allows for changes, additions, or deletions to the database records as needed. It is to be understood that the database 162 may store information in any of a number of ways, including one or more databases having one or more tables. Furthermore, the logical arrangement and relationship of such data and/or tables within a database may take any of a number of forms.

As will be discussed in further detail, the database 162 has several database components including, but not limited to, a quadruple-blind clinical studies database component 164, a medical practice database component 166, a patient database component 168, a compensation database component 170, a unique identifiers database component 172, a BES database component 174, a protocol database component 176, and a verification database component 178. All the database components are readily accessible by the processor 160 and are searchable by key words or as deemed appropriate. The processor 160 also provides a communications component 175 which allows for direct electronic communications between the BES 140, the medical practices 104, the medical director 110, and the board of medical advisers 126 via the communications component 175, however, the board of medical advisers 126 has no communication with the BES 140 to preclude any direct or indirect influence of the quadruple-blind clinical study. Equally important, the board of medical advisers 126 is further responsible for reviewing the protocol; ensuring that the medical practice 104 and medical researchers participating in the study are in compliance with state and federal licensing requirements; and approving the medical practice 104 and protocol 180 of a respective quadruple-blind clinical study. The database 162 is structured to efficiently allow for the searching of various data attributable to the patients 106, the medical practices 104, and the quadruple-blind clinical study eligibility requirements associated with a particular drug or device, which may or may not be stored in the protocol 180 of a respective study.

The quadruple-blind clinical studies database component 164 may include, but is not limited to, the age and gender of the prospective or desired patients, their height, weight, genetic characteristics including specific DNA samples or markers, blood pressure ranges, blood sugar levels, the sponsor of the study, and the like. In like manner, the quadruple-blind clinical studies database component 164 may also include, but is not limited to, the roles and identities of the subject, investigator, evaluator, and data analysts of the quadruple-blind clinical study. Yet, it is to be appreciated that the identities of the subject, investigator, evaluator, and data analysts of a quadruple-blind clinical study are concealed through the use of assigned unique identifiers to provide anonymity, reduce bias, and foster more accurate data findings.

The medical practice database component 166 includes, but is not limited to, the practice areas of the medical researcher or their corresponding hospital, the number of patients in their practice, the location of their practice and the like.

The patient database component 168 includes information about all of the patients associated with a particular medical practice 104 and includes their specific height, weight, age, any particular genetic markers, medical history, or the like. And component 168 may include key words associated with a patient's medical history including dictations prepared by the medical professional; lab, radiology and pathological reports; blood work panels and other appropriate information.

The compensation database component 170 includes a module that is adapted to store, update, and distribute compensation to various participants—in accordance with 42 USCS § 1320a-7b and section 1877 of the Social Security Act (hereafter “Act”)—in a quadruple-blind clinical study, based on a set of decision rules which the medical director 110 inputs and loads into component 170. For example, in one embodiment the decision rules are implemented via software, hardware, firmware, or a combination thereof, to distribute payments to a medical researcher based on a flat fee per month, with such information electronically stored in the compensation database component 170. The decision rules can be programmed to implement various methods with regards to compensating a medical researcher, the board of medical advisers 126, etc. Furthermore, the decision rules may be implemented such that the flat fee may be based on the amount of hours a medical researcher has spent on a patient's data entry, utilizing a client terminal 11, that is enrolled in a quadruple-blind clinical study. In an alternate embodiment, the compensation database component 170 may compensate a medical researcher, based on a set of decision rules as entered into component 170 by a medical director 110, on a determination of the average hourly rate earned for similarly situated practitioners based on a 40-hour work week pursuant to fair market value. In an illustrative embodiment, the compensation database component 170 may compensate a chiropractor, for a disc desiccation study, based on the average hourly rate of a chiropractor as calculated by the American Chiropractic Association. It is to be appreciated that different studies, reports, and data may be used in the calculation of the average hourly earning rate of a medical researcher in the United States as determined by area of practice by the physician.

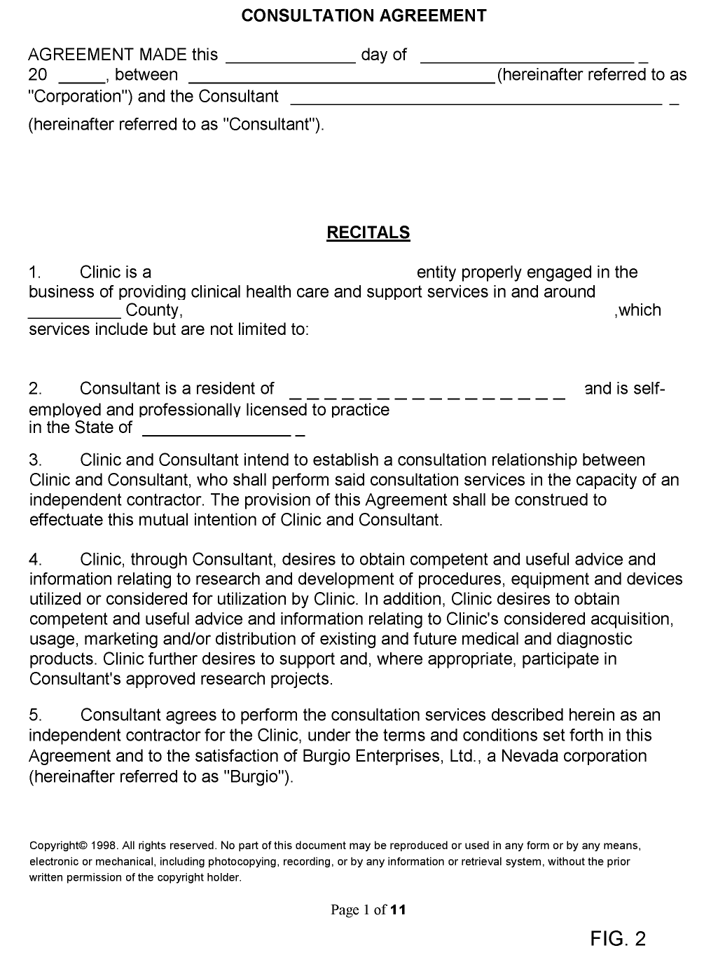

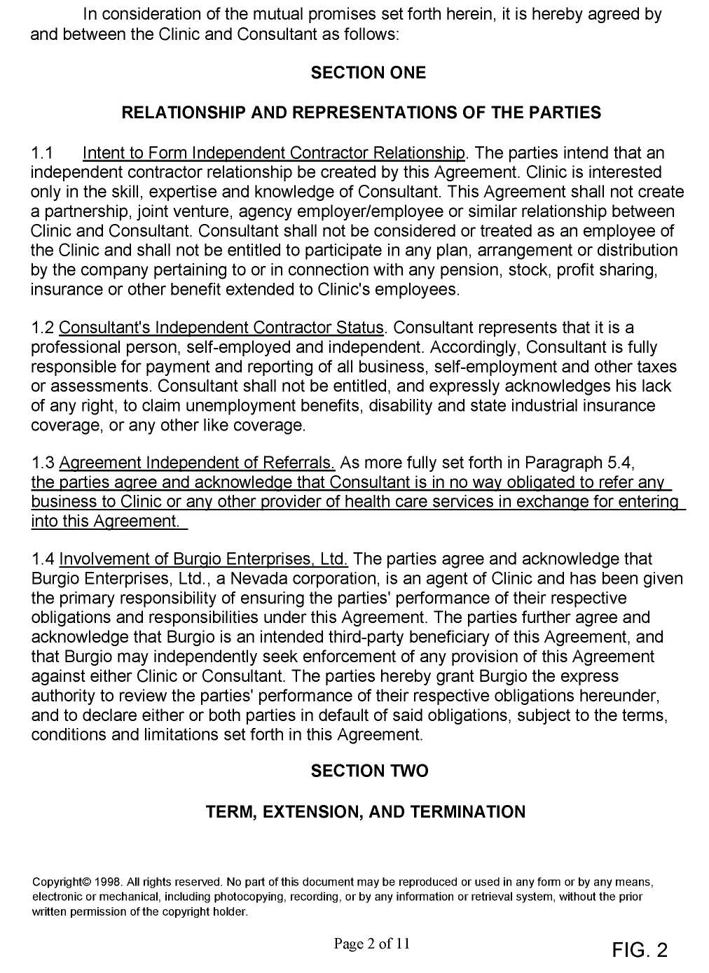

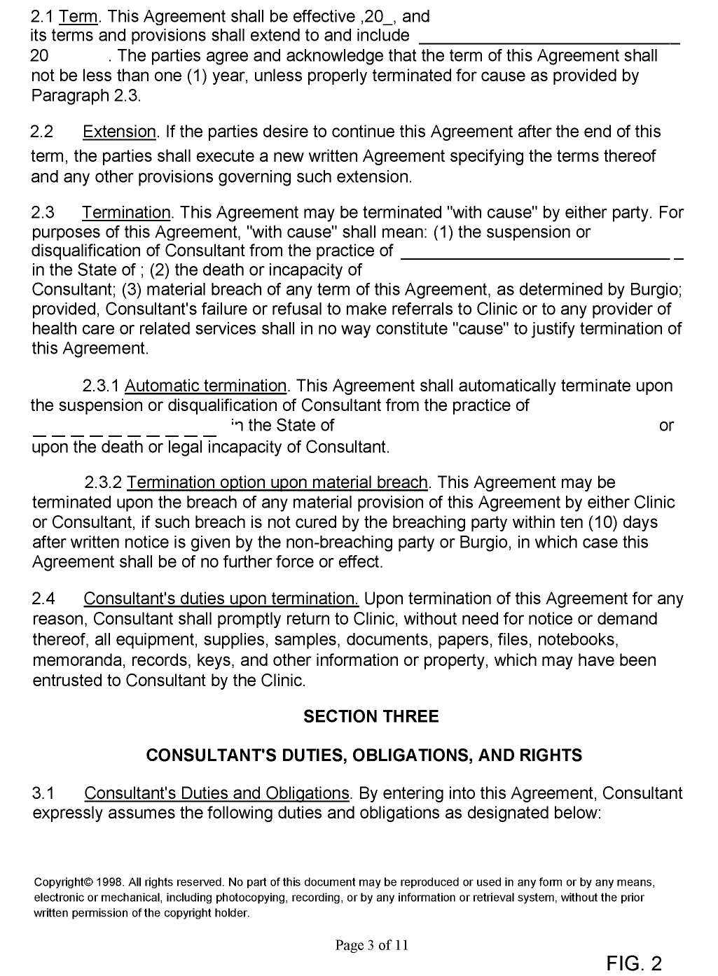

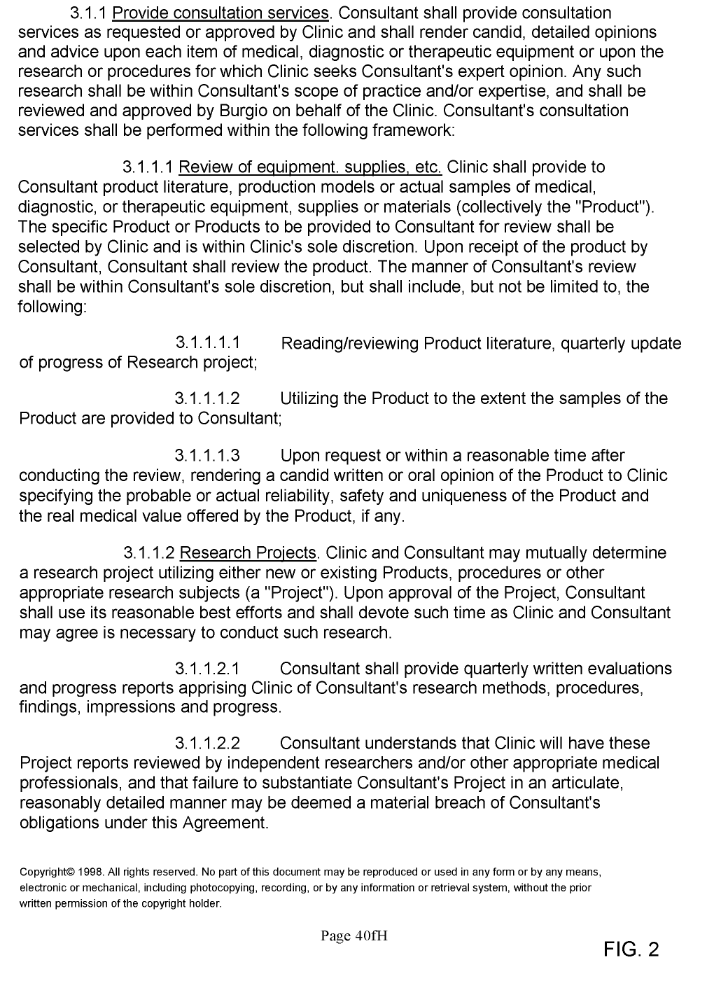

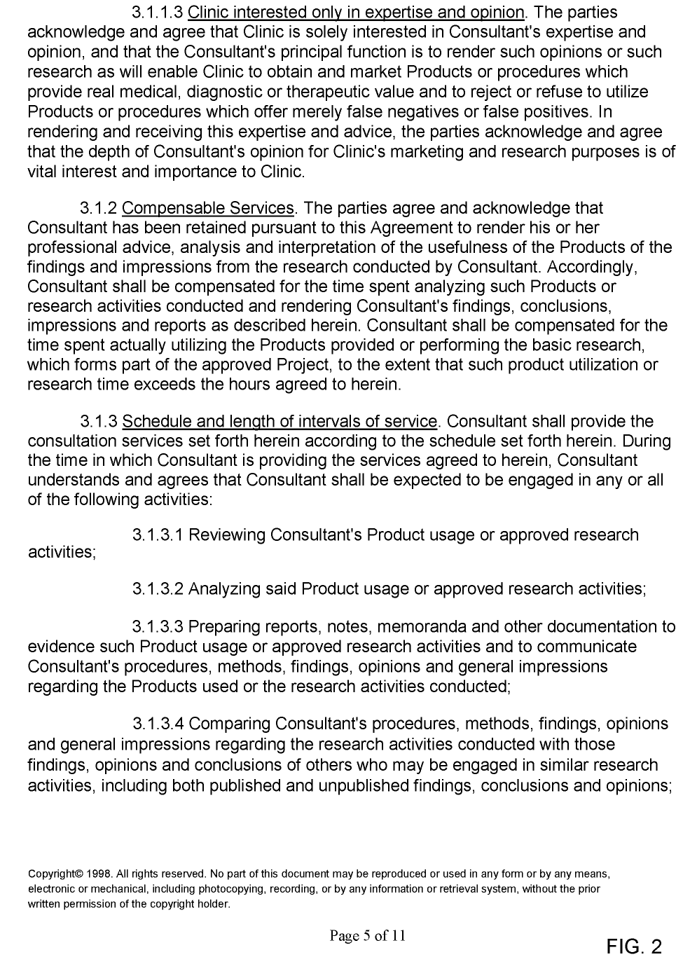

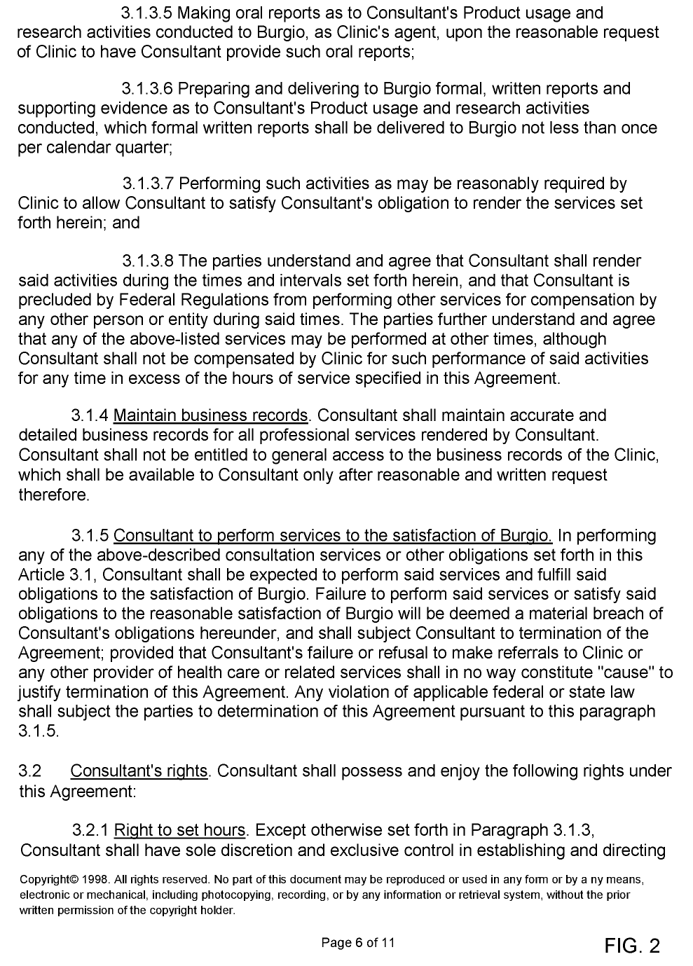

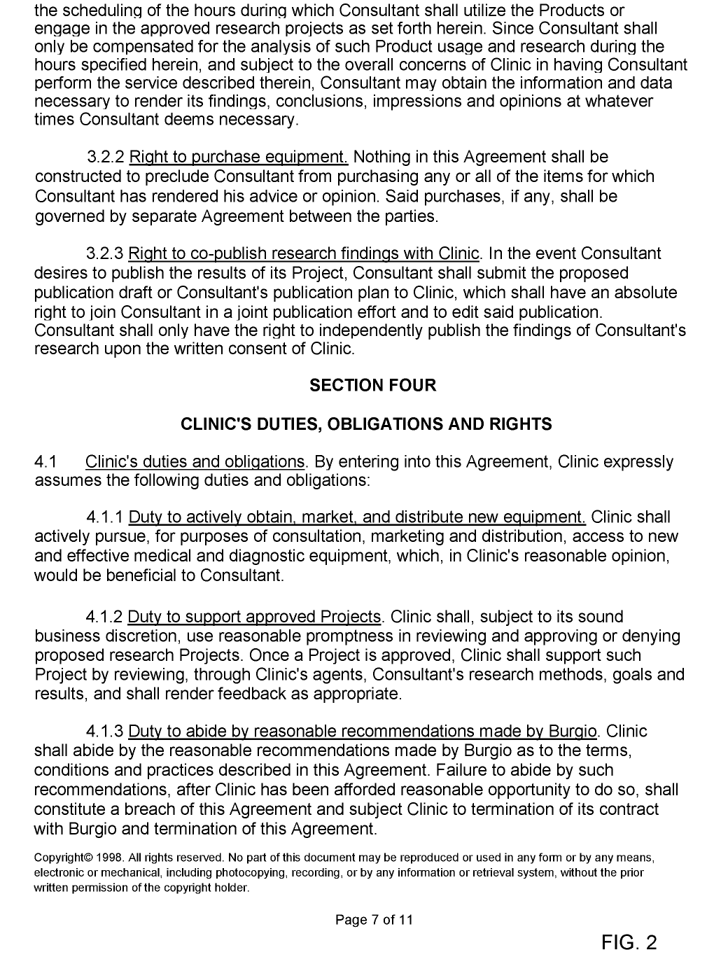

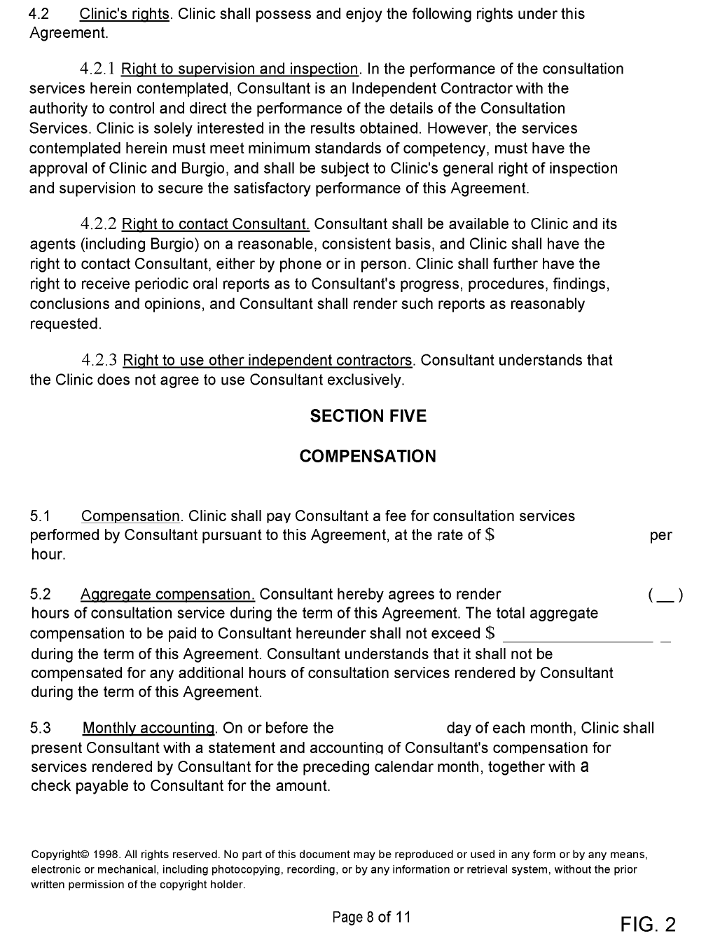

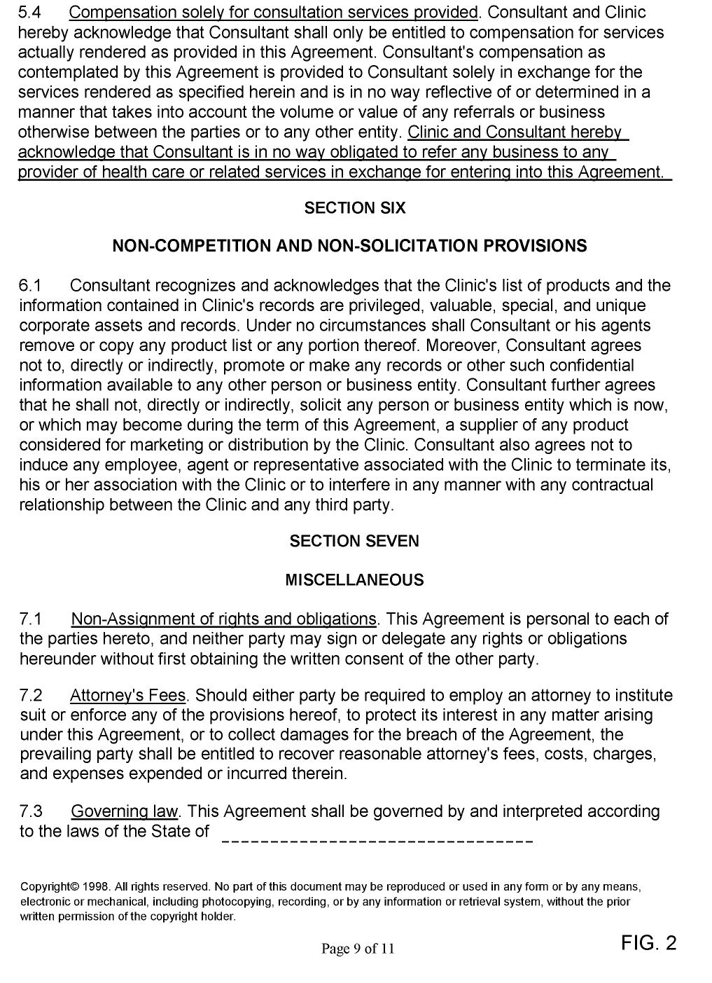

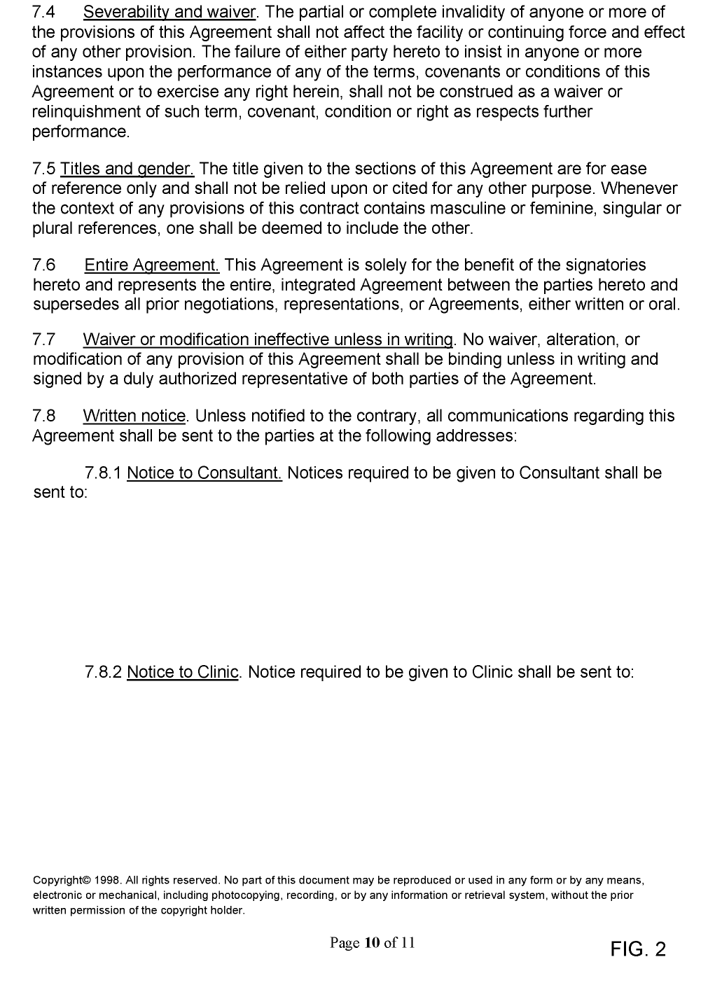

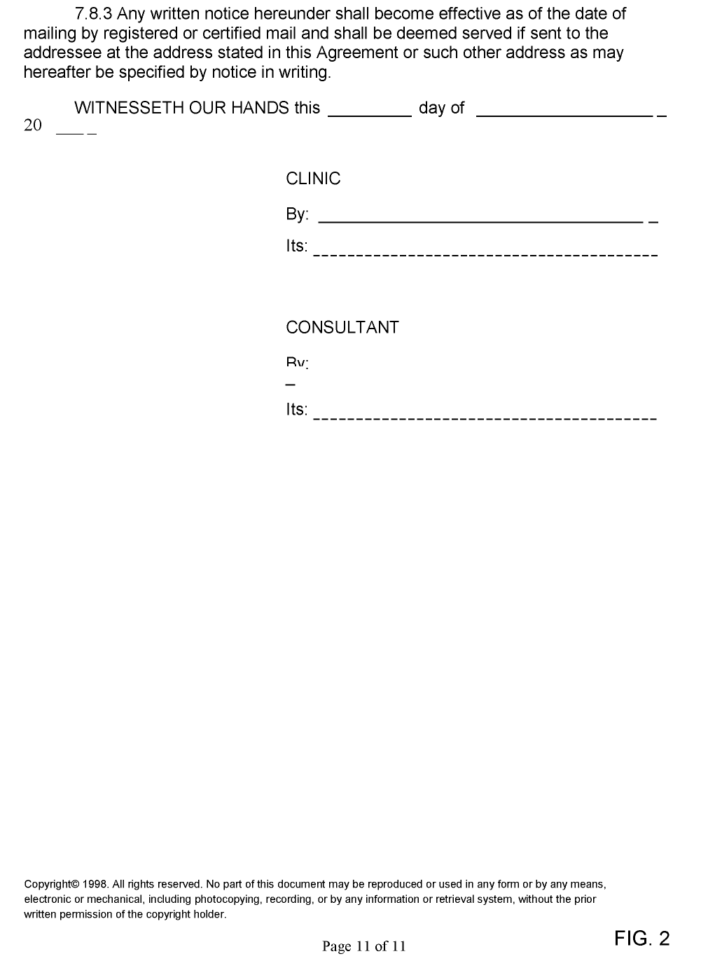

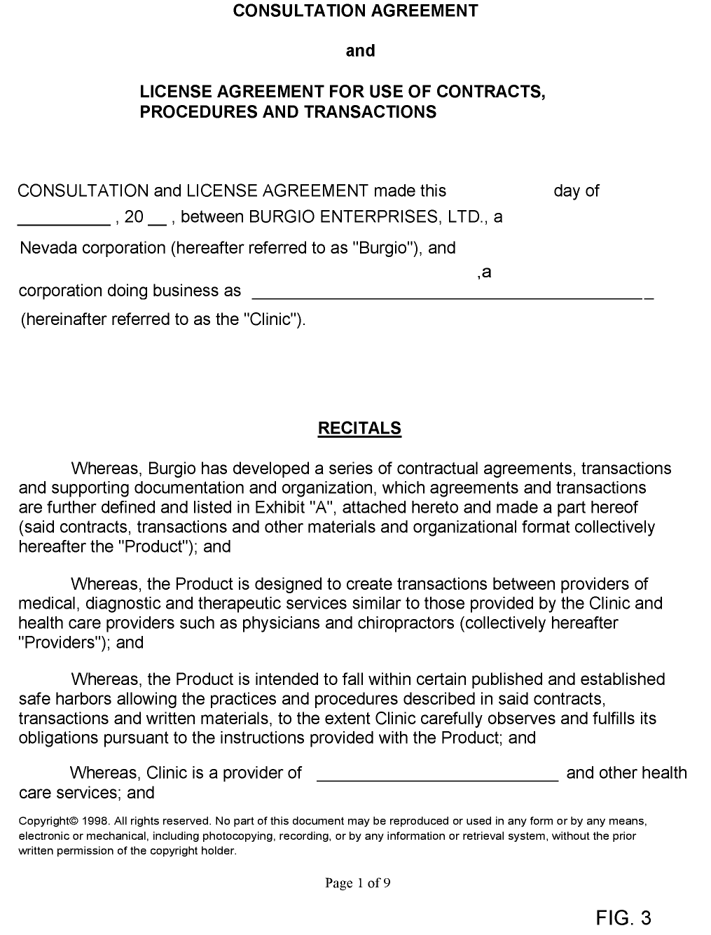

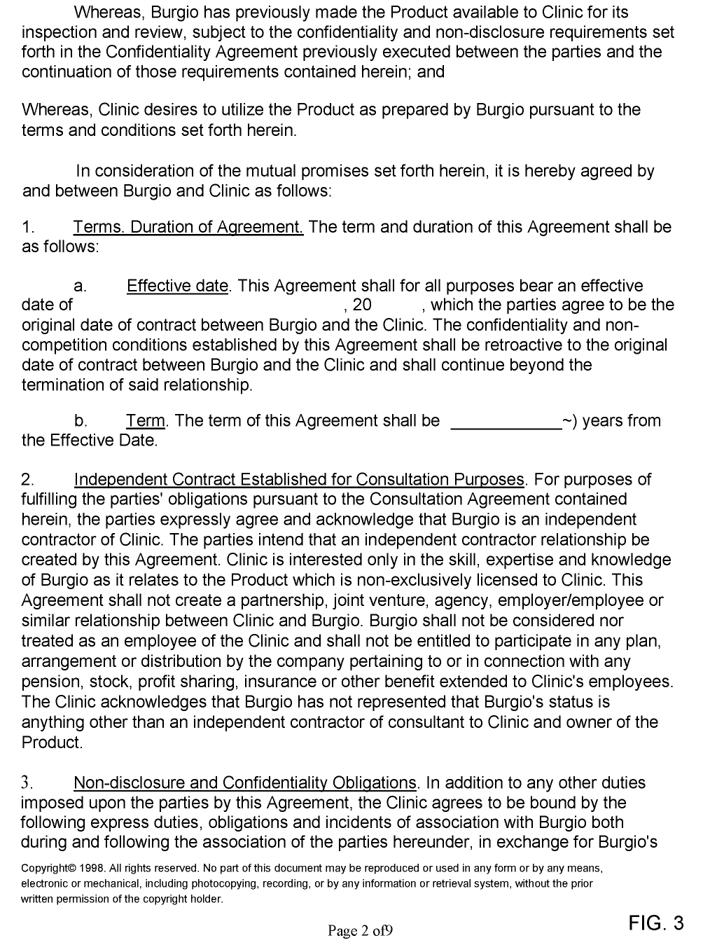

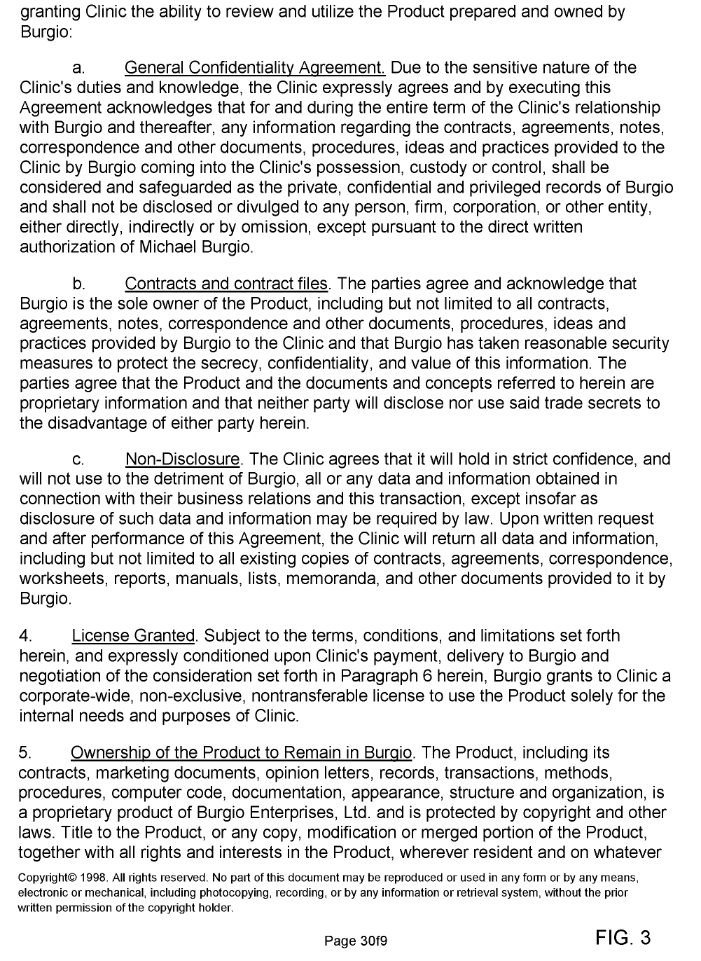

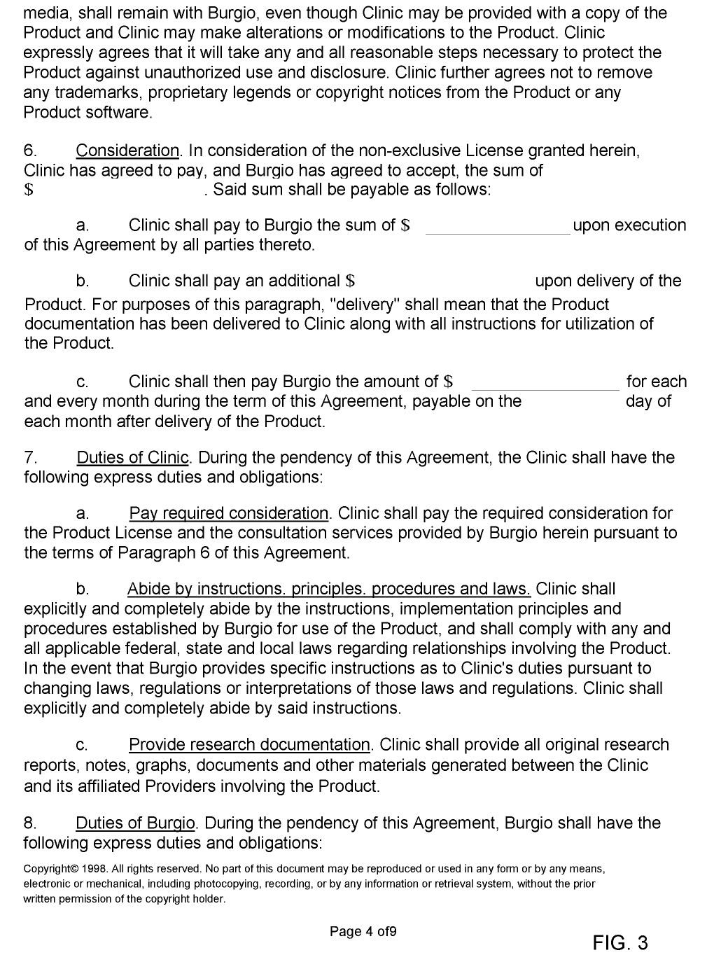

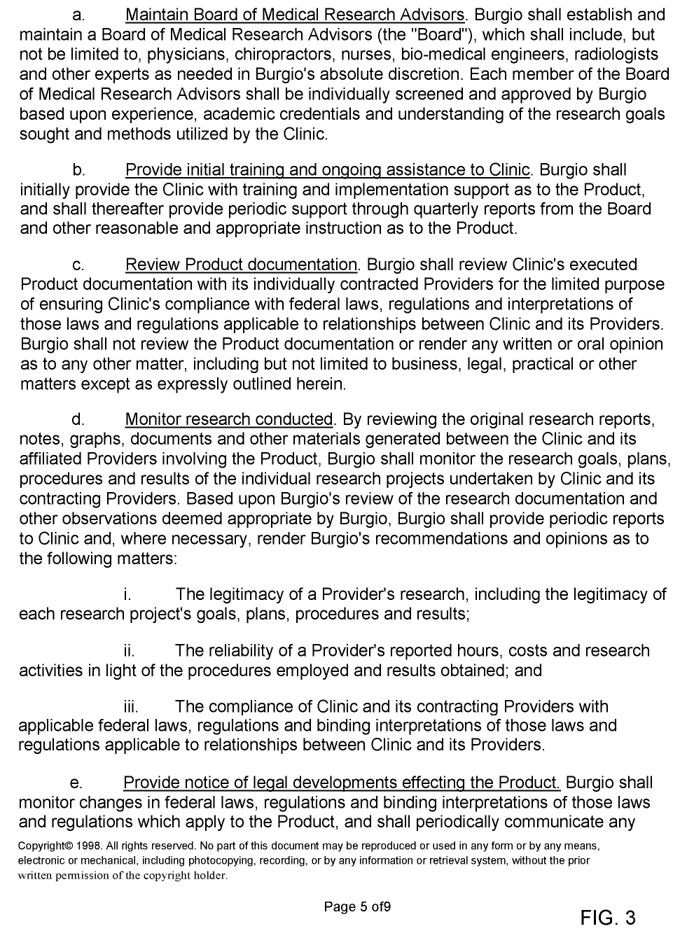

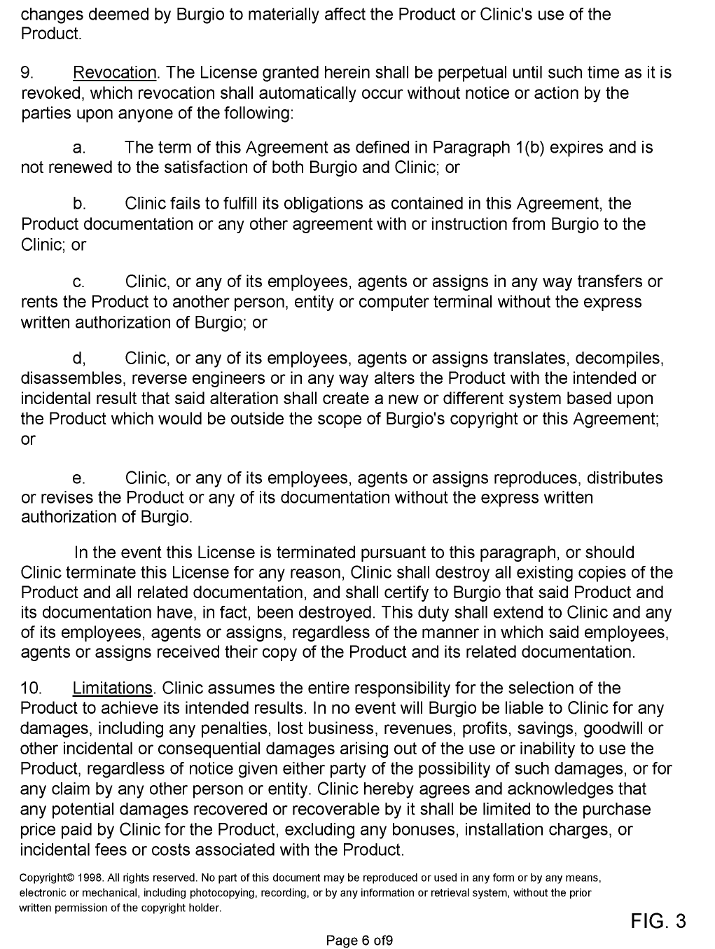

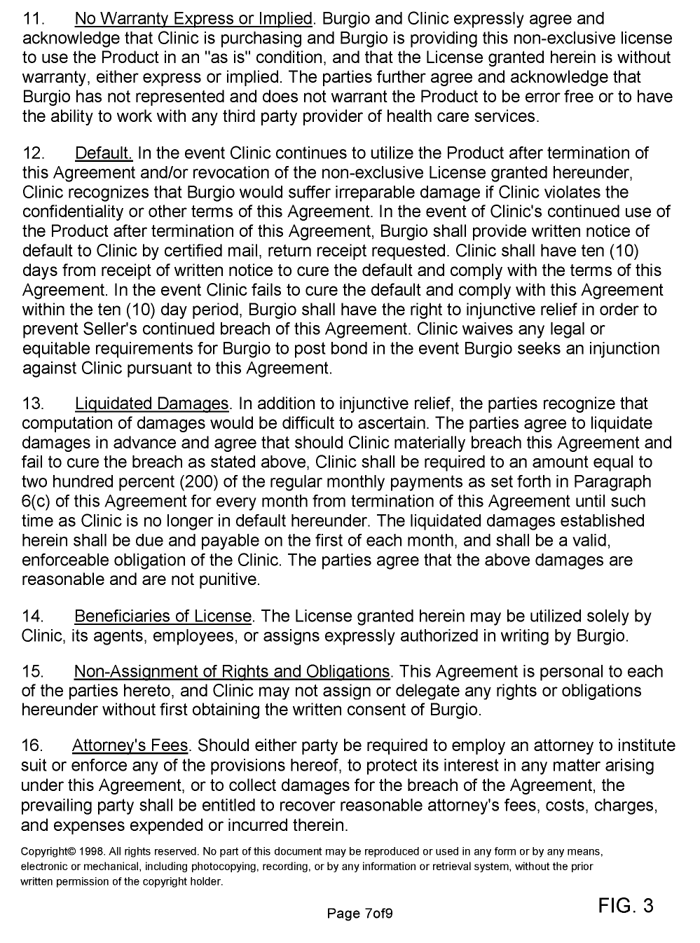

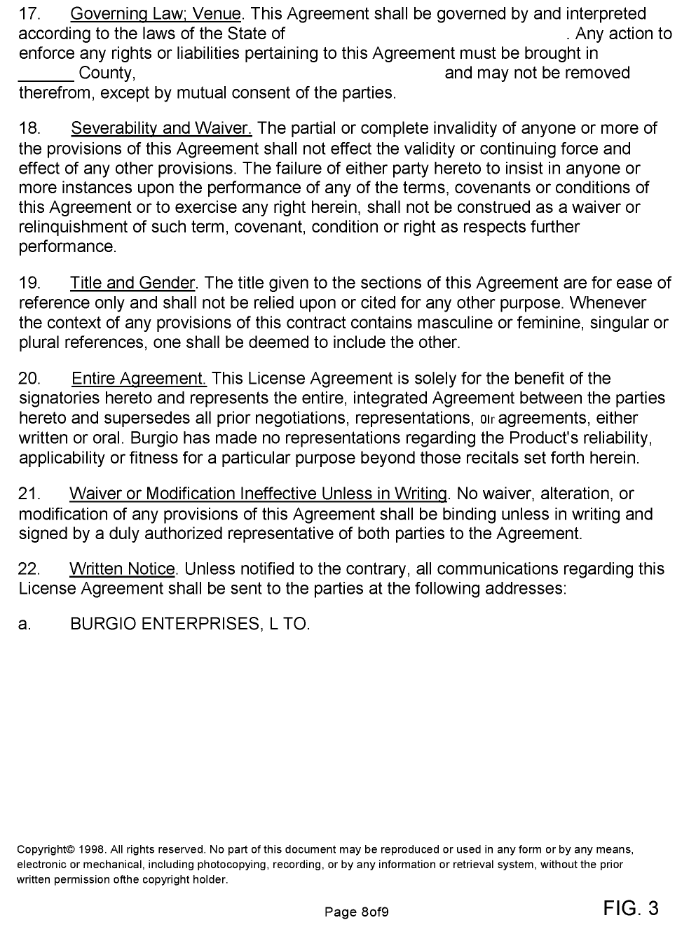

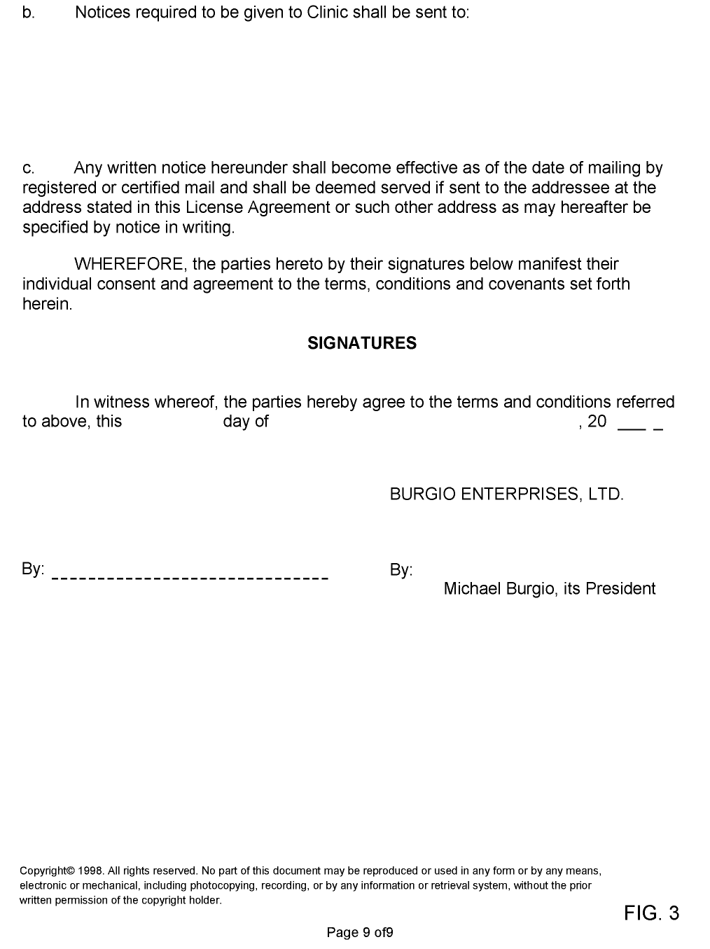

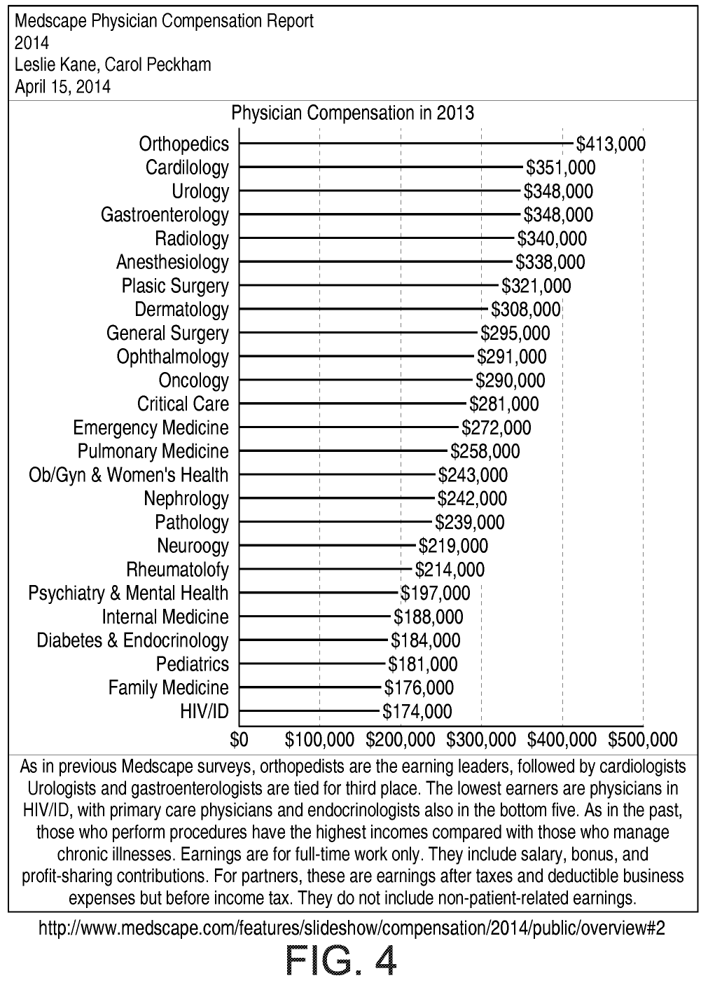

Referring now to FIG. 2, in another illustrative example, the annual Medscape

Physician Compensation Report may be used, in one embodiment, to implement the set of decision rules for calculating the average hourly rate of medical researchers participating in a particular quadruple-blind clinical study. http://www.medscape.com/features/slideshow/compensation/2014/public/overview#2. Thereafter, the medical director 110 may input the set of decision rules via a client terminal 114 in order for the system 100 to utilize the compensation database component 170 to transfer compensation to a medical researcher. In addition, the average hourly rate may be multiplied per patient with regards to the quadruple-blind clinical study to accommodate multiple patients supervised by the medical researcher or referred by a medical researcher. Medical practices 104 may be compensated for both referring patients 106 to a quadruple-blind clinical study or for conducting said study at said medical practice 104.

However, it is to be appreciated that in an alternate embodiment, the compensation database component 170 may compensate various participants of the study according to a consultation agreement, wherein the electronic version of said agreement would be stored in component 170. Further, the consultation agreement will satisfy the seven safe harbor elements as codified in 1128B of the Act. That section provides:

As used in section 1128B of the Act, “remuneration” does not include any payment made by a principal to an agent as compensation for the services of the agent, as long as all of the following seven standards are met—
(1) The agency agreement is set out in writing and signed by the parties. The agency agreement covers all of the services the agent provides to the principal for the term of the agreement and specifies the services to be provided by the agent.
(2) If the agency agreement is intended to provide for the services of the agent on a periodic, sporadic or part-time basis, rather than on a full-time basis for the term of the agreement, the agreement specifies exactly the schedule of such intervals, their precise length, and the exact charge for such intervals.
(3) The term of the agreement is for not less than one year.
(4) The aggregate compensation paid to the agent over the term of the agreement is set in advance, is consistent with fair market value in arms-length transactions and is not determined in a manner that takes into account the volume or value of any referrals or business otherwise generated between the parties for which payment may be made in whole or in part under Medicare, Medicaid or other Federal health care programs.
(5) The services performed under the agreement do not involve the counselling or promotion of a business arrangement or other activity that violates any State or Federal law.
(6) The aggregate services contracted for do not exceed those which are reasonably necessary to accomplish the commercially reasonable business purpose of the services.

For purposes of the above paragraph, an agent of a principal is any person, other than a bona fide employee of the principal, who has an agreement to perform services for, or on behalf of, the principal.

As such, the consultation agreement, in an exemplary embodiment, may be between the medical practice 104 and a medical researcher 104 for conducting the study or for patient referrals provided by the medical researcher 104 or medical practice 104. The medical practice 104 may compensate the medical researcher a consultant fee for consultation services based on a predetermined fixed hourly rate pursuant to a written legal instrument. In an alternate embodiment, the decision rules may compensate a participant of a quadruple-blind clinical study based on a set of decision rules approved by the Department of Health & Human Services—more specifically—the Office of Inspector General, the Guidance Branch Office, which is the federal authority that is tasked with policing any kind of contractual arrangements between licensed physicians and businesspersons.

Equally important, the compensation database component 170 may further utilize a set of decision rules such that participants of a quadruple-blind clinical study are compensated pursuant to written instruments, including but not limited to, license agreements, referral agreements, personal services agreements, and the like. The compensation database component 170 may further utilize the network 118 to distribute payments electronically via a third party payment processor, e.g., Paypal Inc. In addition, the compensation database component 170 compensates participants for preparation of reports/medical papers on a quarterly and yearly basis depending on the duration of the quadruple-blind clinical study; activities performed by participants of the study within the medical practice; mailing charges; data analysis of the study to determine trends; patient referral; complete data analysis once the quadruple-blind study has been completed, and the like.

The unique identifiers database component 172, is operative to assign, store, and update unique identifiers, in alphanumeric form. The identifiers are assigned to each quadruple-blind clinical study in the system 100, and to each board of medical advisers 126 corresponding to said quadruple-blind clinical study and formed by the medical director 110, to each medical researcher, to each medical practice 104, and to each patient 106. In one embodiment, the medical researcher 110 assigns the unique identifiers to the respective quadruple-blind clinical study, board of medical advisers 126, and a unique identifier for the medical researcher and medical practice 104 collectively. Thereafter, the medical researcher assigns a unique identifier to the patients 106, as a result, the true identity of the patient 106 is exclusive to the medical researcher.

The BES database component 174 may include a plurality of contract research organizations and keep an accounting for the medical director 110 of which BESs 140 received which patients 106 and how much funding the BES 140 has transferred to the medical director 110. And, at least one client terminal 114 may be linked to the database 162 via the communication component 175 to allow for searching of the various database components and linking to other computers via a network 118 (or internet) utilizing a

User Interface that will be used to input data onto a server that will be secured with a router 113. In a preferred embodiment, the Cisco 4451-x Integrated Services Router may be used, in which the Cisco router will then create VPH connections throughout the logged session. The VPH is performed by using Cisco Group Encrypted Transport VPN (GET VPN), which eliminates the need for compromise between network intelligence and data privacy in private WAN environments. Service providers can offer managed encryption without a provisioning and management nightmare since GET VPN simplifies the provisioning and management of VPN. GET VPN defines a new category of VPN, one that does not use tunnels. Thus, allowing site to site data transfer without being compromised.

Networks have become critical strategic assets and lifelines for running successful enterprises. Today's networks not only support critical applications, but also support voice and video infrastructures. Applications and technologies, such as distributed computing and voice and video over IP, now require instantaneous branch-to-branch communication. Because of these requirements, the traditional hub-and-spoke topology of enterprise networks is no longer sufficient. Enterprises must implement the any-to-any connectivity model provided by IP virtual private networks (VPNs) and virtual private LAN services (VPLS) networks. Although IP VPN and VPLS services built with Multiprotocol Label Switching (MPLS) separate enterprise traffic from the public Internet to provide some security, in recent years government regulations, such as Health Insurance Portability and Accountability Act (HIPAA), Gramm-Leach Bliley Act (GLBA), and Payment Card Industry Data Security Standard (PCI DSS), mandate encryption even over private IP networks.

Cisco IOS offers several IP security (IPsec) tunnel-based encryption solutions (for example, Site to Site IPsec, IPsec/generic routing encapsulation (GRE), and Dynamic Multipoint VPN (DMVPN) that can be deployed over an MPLS VPN, VPLS or shared IP networks. Traditional tunnel-based encryption solutions are point-to-point. To provide a true full mesh or even dense partial mesh of connectivity, tunnel-based solutions require the provisioning of a complex connectivity mesh. Such a complex mesh not only has higher processor and memory requirements, but is difficult to provision, troubleshoot, and manage. Some provisioning overhead can be reduced using DMVPN. However, DMVPN requires overlaying a secondary routing infrastructure through the tunnels, which results in suboptimal routing while the dynamic tunnels are built. The overlay routing topology also reduces the inherent scalability of the underlying IP VPN network topology.

Traditional point-to-point IPsec tunneling solutions suffer from multicast replication issues because multicast replication must be performed before tunnel encapsulation and encryption at the IPsec CE (customer edge) router closest to the multicast source. Multicast replication cannot be performed in the provider network because encapsulated multicasts appear to the core network as unicast data. Cisco's Group Encrypted Transport VPN (GET VPN) introduces the concept of a trusted group to eliminate point-to-point tunnels and their associated overlay routing. All group members (GMs) share a common security association (SA), also known as a group SA. This enables GMs to decrypt traffic that was encrypted by any other GM. (Note that IPsec CE acts as a GM.) In GET VPN networks, there is no need to negotiate point-to- point IPsec tunnels between the members of a group, because GET VPN is “tunnel-less.”

Once the user has loaded the User Interface for access to the software they will then be connected to the above-mentioned Cisco Router with encryption from start to the end. Each user will need to provide proper credentials to access the secured network. Once a user has connected to the router, the user will automatically gain access to the data Input Interface where they will securely enter data as required by the study. The User Interface does not store information related to the name or location of the user, as assigned ID numbers are used to identify the study, respective Board of Medical Advisors, respective clinic, and respective patients. As such, multiple levels of anonymity are disclosed, which comprise the quadruple-blind study.

The protocol database component 176, is configured to store, update, and retrieve a plurality of quadruple-blind clinical study protocols. The medical director 110 will design a protocol for a corresponding quadruple-blinded clinical study. Further, the protocol may be a predefined written procedural method in the design and implementation of a quadruple-blinded clinical study. As indicated above, the protocol may describe how a quadruple-blind clinical study will be conducted (the objective(s), design, methodology, statistical considerations, eligibility requirements, and organization of the quadruple-blind clinical study,) and ensures the safety of the patients and integrity of the data collected. Thereafter, the medical director may send the protocol, via the communications component 175, to the sponsor 120 and the corresponding medical practice 104. The sponsor 120 and the medical practice 104 must approve the protocol 120. The protocol 120 will then be field-tested to ensure the protocol 120 is within good medical practices pursuant to state and federal requirements, and to determine whether the protocol is viable, achievable, and plausible. The foregoing attributes are all stored in the protocol database component 176, via the processor 160, utilizing various tables and may be displayed on client terminals 114.

The verification database component 178 is operative to be in communication with the communications component 175 and further adapted to verify a medical researcher's compliance with state and federal licensing requirements, wherein said communications component is further configured to electronically notify, in real time, the component 178 of said research physician's noncompliance with state and federal licensing requirements via said verification database component. In turn, the medical director 110 and board of medical advisers 126 may be alerted, by the communications component 175, to a change in a medical researcher's compliance and good standing with state and federal regulations with respect to the practice of medicine.

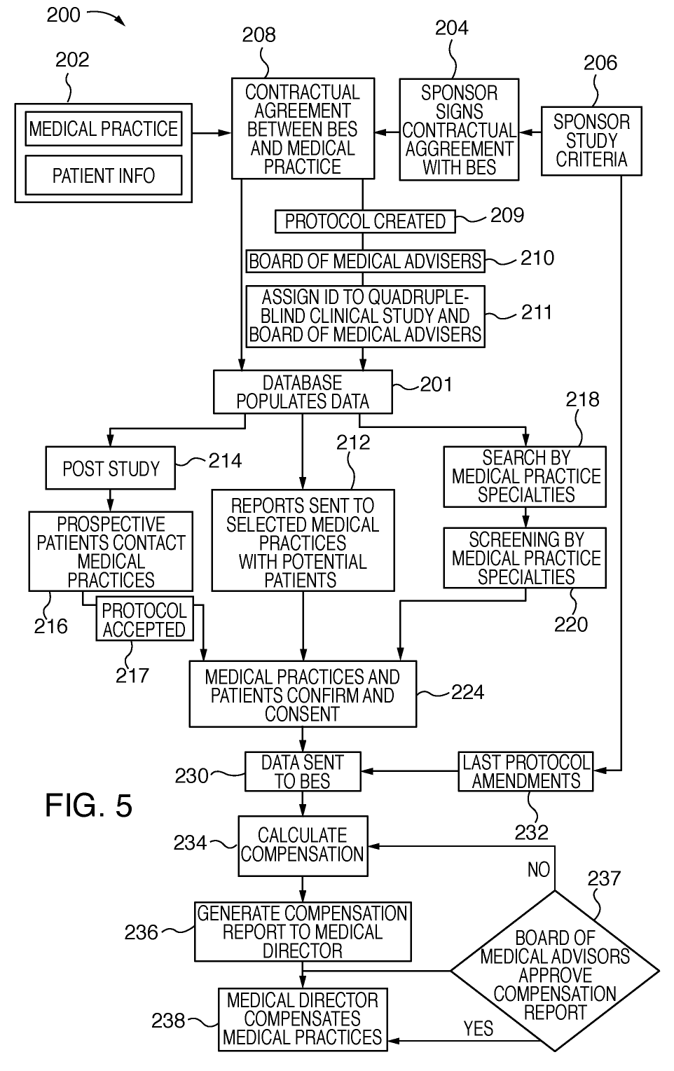

Referring now to FIG. 5, the processes which are used in implementing the system 100 is designated generally by the numeral 200. The process 200 utilizes the following steps for matching suitable patients to quadruple-blind clinical studies. In particular, at step 201, the database populates data inputs made to the system 100 by a medical director 110, medical practice 104, medical researcher (not shown), sponsor 120, or a client terminal 114—the processor 160. Moreover, at step 202, the medical practice 104 submits registration information regarding the medical practice, the suitable patients for a particular quadruple-blind clinical study, and the like, for loading into the database 162, via the processor. At a step 204, a contractual agreement is signed between the sponsor 120 and the BES 140 pertaining to the duties and obligations to be performed by both the sponsor 120 and the BES 140 in connection with a quadruple-blind clinical study. At a step 206, the sponsor 120 is responsible to identify the specific quadruple-blind clinical study, the general outline of what it is trying to be accomplished, and the quadruple-blind clinical study criteria.

To reiterate, a quadruple-blind clinical study is a study in which the subjects, investigator(s), evaluator, and the data analysts remain blinded. In one embodiment, the patients 106 of a quadruple-blind clinical study are the subjects; the medical researcher is the investigator; the board of medical advisers 126 is the evaluator; and the medical director 110 is the data analyst. As noted previously, the sponsor 120 needs to obtain test data regarding their respective drugs or devices to comply with Food and Drug Administration regulations and to assure the general consuming public that the new drugs or devices which they have developed are safe for use. Accordingly, in exchange for test data obtained by these quadruple-blind clinical studies, sponsors will pay a predetermined amount of money to a BES 140, via a contractual agreement, to be distributed by the medical director 110 to the appropriate parties Similarly, in exchange for patient referrals to these quadruple-blind clinical studies, the medical practices 104 and the medical researchers will receive compensation pursuant to a contractual agreement, such as a license agreement, to be distributed by the medical director 110.

Upon completion of step 201, several methodologies may be employed for obtaining the necessary patients 106 for the quadruple-blind clinical study requested by the sponsor 120. At step 208, in exchange for patient referrals to these quadruple-blind clinical studies, the medical practices 104 and the medical researchers will enter into a legally binding contractual agreement, such as a license agreement, or the like.

In step 209, according to one embodiment, the BES 140 will assign a medical director 110 for a corresponding quadruple-blind clinical study who will then design a preliminary protocol 180 for a corresponding quadruple-blind clinical study, which must be approved by both the sponsor 120 and the medical practice 104. Further, in step 210, the protocol is field-tested to ensure that the protocol is viable.

In step 210, the medical director 110 will then establish a board of medical advisers 126 to provide oversight. Then, in step 211 the medical director assigns a unique alphanumeric identifier to the quadruple-blind clinical study and to the board of medical advisers 126—to maintain anonymity and reduce biased, skewed data findings.

In a preferred embodiment, the medical director 110 generates a report, at step 212, wherein the processor 175 matches the quadruple-blind clinical study information, protocol, and the like, in the database component 162 with the medical practice database component 166 stored on the database 162. In particular, the matching report will also include a listing of the patients 106 that potentially fulfill the requirements of the quadruple-blind clinical study requested by the sponsor 120 and said report will be sent electronically to the medical practice 104 and the medical director 110, via the communications component 175. However, it should be appreciated that the report of step 212 may also contain a list of patient referrals, which match the protocol of a study, submitted by medical practices 104 or medical researchers. Alternatively, at step 214, the quadruple-blind study eligibility requirements and protocol may by listed on an internet site by the system 100, or alternatively advertised in professional journals, newspapers, or magazines that reach the target audience for the quadruple-blind clinical study. At step 216, prospective patients may then contact the medical practice 104 conducting the study, directly, and submits their medical information via the communications component 175 associated with the processor 160 through a client terminal 114. At step 217, the preliminary protocol must be accepted by the board of medical advisers 126, and the medical practice 104 will then approach potential patients for said quadruple-blind clinical study—the medical practice 104 may also explain the goals of the study, the protocol, and the parameters of compensation agreement that governs.

In an alternative embodiment, a medical practice 104 or patient 106, may search the system's website, at step 218, by inputting particular search words that afflict a particular patient 106. This may be done manually by just reviewing a listing of the searches or it may be done by inputting key words. At step 220, the medical practice 104, or medical researcher, screens and flags the quadruple-blind clinical studies that may be of assistance to their particular patients 106 and such information is stored and updated in the database 162.

In any event, upon completion of step 212, the communications component 175 of the system 100 is utilized and the medical practices 104 confirm and obtain the consent of the prospective patients to affirm that they match the criteria requested by the sponsor and to obtain all the necessary release forms from the patients so that they understand the risks and potential benefits of being part of the quadruple-blind clinical study, in a step 224, and unique identifiers are assigned to the confirmed patients 106 of the study and the medical practices 104 associated therein. Upon completion of step 224, the appropriate data is sent to the BES 140 and upon receipt of the appropriate number of patients for the study, the study may be commenced. It will be appreciated that, at step 232, the specific study protocol 180 is provided by the sponsor 120 and any final changes or adjustments made between the contracting step and the step of initiating the actual study is likely confirmed and verified.

At step 234, the quadruple-blind clinical study is initiated and the system 100 calculates compensation, as described above, to various participants of a quadruple-blind clinical study, including the medical researchers that conducted the study, the medical researchers who referred patients for the study but otherwise did not participate in said study, medical practices 104, BESs 140, board of medical advisers 126, and other entities involved. Once the quadruple-blind clinical study is performed and the compensation has been calculated, a compensation report is generated and transmitted to the medical director 110, at step 236, by the system 100 and sent to the appropriate BES 140.

At a step 237, the compensation report is then likely approved by a board of medical advisers 126. Alternatively, if the bill is not approved by the board of medical advisers 126, then the compensation will have to be re-calculated to fit the financial constraints and budget of the sponsor 120, whereby another medical practice 104 within the budget of the sponsor 120 may be utilized. At step 238, the medical director 110 will compensate the appropriate parties—the system 100 may possess the necessary hardware, software, firmware, and any combination thereof, to transfer payment electronically.

As disclosed herein, the example processes of FIG. 5. may be embodied as machine-accessible instructions and executed by, for example, the processor 160. A processor, a controller and/or any other suitable processing device may be used, configured and/or programmed to perform the example processes of FIG. 5. For example, the process of FIG. 5 may be embodied in coded instructions stored on a tangible computer-readable medium. Machine-readable instructions comprise, for example, instructions that cause a processor, a computer and/or a machine having a processor to perform one or more particular processes. Alternatively, some or all of the components of the example process of FIG. 5 may be implemented using any combination(s) of ASIC(s), PLD(s), FPLD(s), FPGA(s), fuses, discrete logic, hardware, firmware, etc. Also, some or all of the components of the example process of FIG. 5 may be implemented manually or as any combination of any of the foregoing techniques, for example, any combination of firmware, software, discrete logic and/or hardware. Further, many other methods of implementing the example operations of FIG. 5 may be employed. For example, the order of execution of the blocks may be changed, and/or one or more of the blocks described may be changed, eliminated, sub-divided, or combined. Additionally, the blocks of any or all of the components of the example process of FIG. 5 may be carried out sequentially and/or carried out in parallel by, for example, separate processing threads, processors, devices, discrete logic, circuits, etc.

While the invention has been described in connection with a preferred embodiment, it is not intended to limit the scope of the invention to the particular form or methodology set forth, but it is intended to cover such alternatives, modifications, and equivalence as may be included within the spirit and scope of the invention as defined by the attached claims. While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

It should be understood that the various examples of the apparatus(es) disclosed herein may include any of the components, features, and functionalities of any of the other examples of the apparatus(es) disclosed herein in any feasible combination, and all of such possibilities are intended to be within the spirit and scope of the present disclosure. Many modifications of examples set forth herein will come to mind to one skilled in the art to which the present disclosure pertains having the benefit of the teachings presented in the foregoing descriptions and the associated drawings.

Therefore, it is to be understood that the present disclosure is not to be limited to the specific examples presented and that modifications and other examples are intended to be included within the scope of the appended claims. Moreover, although the foregoing description and the associated drawings describe examples of the present disclosure in the context of certain illustrative combinations of elements and/or functions, it should be appreciated that different combinations of elements and/or functions may be provided by alternative implementations without departing from the scope of the appended claims.

Embodiments:

1. A system for recruiting eligible patients for enrollment in a quadruple-blind clinical study, via a network, from a plurality of medical practices, wherein said quadruple-blind clinical study being conducted by one of a plurality of clinical study entities, comprising:
a database component operative to maintain and to store a medical practice database component having a plurality of medical practices each identified by a corresponding specialty, a quadruple-blind clinical studies database component having a plurality of quadruple-blind clinical studies, a protocol database component having a plurality of quadruple-blind clinical study protocols, a patient database component having a plurality of patient records, each of said plurality of patient records identifying a patient and the patient's association with at least one of said plurality of medical practices in said medical practice database component, a unique identifiers database component having a plurality of unique identifiers, a BES database component including a plurality of contract research organizations, a verification database component coupled to the communications component and further adapted to verify a medical researchers compliance with state and federal licensing requirements, a compensation database component having a plurality of decision rules, wherein said decision rules are implemented to compensate a participant in said quadruple-blind clinical study based on a binding legal written instrument, wherein said binding written legal agreement complies with 42 CFR 1001.952(d);
a communications component, coupled to the plurality of clinical study entities and to the plurality of medical practices through a communications link, to electronically said medical practices in real time to the existence of said clinical studies, to electronically notify said clinical study entities to the existence of said medical practices in real time, to electronically notify said clinical study entities and said medical practices to the existence of patients suitable for enrollment in a quadruple-blind clinical study based upon information from said database components and to receive changes to said database components, to receive by a computer device a plurality of input data from said medical practices via an interactive graphical user interface comprising fields of required data pre-defined by the computer device;
a registration component configured to communicate with said database components utilizing said communications component to present one or more web pages that allow a user to register with said patient database component, by submitting registration information to said patient database component, wherein the registration information includes at least a geographic location of the user, at least one disease condition of interest to the user, contact information, and permission information indicating whether the user wishes to receive electronic notice of said plurality of quadruple-blind clinical studies;
a processor, coupled to said communications component and to said database components, configured to update said database components, to match at least one of said plurality of medical practices with at least one of said plurality of quadruple-blind clinical studies, to query said patient database component to identify data that indicate patients who may be suitable for said quadruple-blind clinical studies, wherein said query includes searching for key words embedded in the patient database records, to match patients suitable for enrollment with said medical studies based upon information from said database components, to generate a report of medical practices matched to said clinical studies, to generate a report of patients matched to said quadruple-blind clinical studies,
to utilize said communications component so as to electronically notify in real time said medical practices associated with said matched patients, and to calculate, utilizing said compensation database component, a fee to be paid to a participant for referral procedures performed in referral of said matched patients to said quadruple-blind clinical study to which said patient is matched;
wherein said communications component sends at least one of said reports over said communications link to said medical practices in said medical practice database to electronically notify in real time said medical practices of said matched patients for referral, and wherein said medical practices locate and refer said matched patients to said quadruple-blind clinical study to which each said patient is matched; and wherein said medical practices electronically notify in real time said clinical study entities of referral of said patients to said medical studies over said communications link utilizing said communication component, and said database components are updated utilizing said processor;

2. The system according to claim 1, wherein each of said plurality of unique identifiers corresponds to:
each of said plurality of quadruple-blind clinical studies, each of said plurality of medical practices,
a medical director,
a board of medical advisers, and
each of said plurality of patient records.

3. The system according to claim 1, further comprising:
a listing component for listing each of said plurality of quadruple-blind clinical studies in said quadruple-blind clinical studies database component, wherein said listing component is further configured to list said quadruple-blind clinical study on an internet site, online professional journals, online newspapers, and online magazines that reach a target audience for the quadruple-blind clinical study.

4. The system according to claim 1, further comprising:
a searching component for searching said quadruple-blind clinical studies database and said medical practice database, wherein said communications component is adaptable to receive queries from said medical practices and said quadruple-blind clinical studies via said searching component.

5. The system according to claim 1, wherein said compensation database component is further operative to maintain an electronic copy of said binding legal written instrument, and wherein said legally binding written instrument includes a contract, license agreement, personal services and management agreement, and consultation agreement.

6. The system according to claim 5, wherein said compensation component is further operative to transfer monetary payment to a medical researcher, wherein said monetary payment is calculated, via a processor, in conjunction with said compensation database component, at a flat fee per month, and wherein said flat fee per month will reflect the amount of hours spent on each of said plurality of patient records by said medical researcher.

7. The system according to claim 6, wherein said compensation database component is further adapted to compensate said medical researcher pursuant to the requirements as set out by said consultation agreement.

8. The system according to claim 6, wherein said compensation database component is further adapted to compensate a referral physician pursuant to the requirements as set out by said consultation agreement.

9. The system according to claim 6, wherein said verification component is further configured to store an electronic version of said legally binding written instrument.

10. The system according to claim 1, wherein said compensation component is further operative to transfer monetary payment to a medical researcher, wherein said monetary payment is calculated, via the processor, based on the average hourly rate of a medical researcher pursuant to a Medscape Physician Compensation Report.

11. The system according to claim 1, wherein said network includes the internet. 12. The system according to claim 1, wherein said network further utilizes a Cisco 4451-x Integrated Services Router.

13. The system according to claim 1, wherein said system can be implemented in Ireland.

14. The system according to claim 1, wherein said clinical study entities includes medical manufacturing corporations in both Asia and Europe.

15. The system according to claim 1, wherein said medical practices are located within the United States.

16. The system according to claim 1, wherein said clinical study entities includes a pharmaceutical or medical device company with a principal place of business in Asia.

17. The system according to claim 2, wherein said medical director is responsible for inputting data into said compensation component.

18. The system according to claim 8, wherein said legally binding instruments are signed in Ireland.

19. The system according to claim 1, wherein said clinical study entities includes a pharmaceutical or medical device company with a principal place of business in Europe.

What is claimed is:

1. A computer-implemented method for recruiting eligible patients for enrollment in a quadruple-blind clinical study, via a network, from a plurality of medical practices, wherein said quadruple-blind clinical study being conducted by one of a plurality of clinical study entities, the method comprising:
storing a medical practice database component having a plurality of medical practices each identified by a corresponding specialty, a quadruple-blind clinical studies database component having a plurality of quadruple-blind clinical studies, a protocol database component having a plurality of quadruple-blind clinical study protocols, a patient database component having a plurality of patient records, each of said plurality of patient records identifying a patient and the patient's association with at least one of said plurality of medical practices in said medical practice database component, a unique identifiers database component having a plurality of unique identifiers, a BES database component including a plurality of contract research organizations, a verification database component coupled to the communications component and further adapted to verify a medical researchers compliance with state and federal licensing requirements, a compensation database component having a plurality of decision rules, wherein said decision rules are implemented to compensate a participant in said quadruple-blind clinical study based on a binding legal written instrument, wherein said binding written legal agreement complies with 42 CFR 1001.952(d);
storing a communications component, coupled to the plurality of clinical study entities and to the plurality of medical practices through a communications link, to electronically said medical practices in real time to the existence of said clinical studies, to electronically notify said clinical study entities to the existence of said medical practices in real time, to electronically notify said clinical study entities and said medical practices to the existence of patients suitable for enrollment in a quadruple-blind clinical study based upon information from said database components and to receive changes to said database components, to receive by a computer device a plurality of input data from said medical practices via an interactive graphical user interface comprising fields of required data pre-defined by the computer device;
storing a registration component configured to communicate with said database components utilizing said communications component to present one or more web pages that allow a user to register with said patient database component, by submitting registration information to said patient database component, wherein the registration information includes at least a geographic location of the user, at least one disease condition of interest to the user, contact information, and permission information indicating whether the user wishes to receive electronic notice of said plurality of quadruple-blind clinical studies; and,
utilizing a processor, coupled to said communications component and to said database components, configured to update said database components, to match at least one of said plurality of medical practices with at least one of said plurality of quadruple-blind clinical studies, to query said patient database component to identify data that indicate patients who may be suitable for said quadruple-blind clinical studies, wherein said query includes searching for key words embedded in the patient database records, to match patients suitable for enrollment with said medical studies based upon information from said database components, to generate a report of medical practices matched to said clinical studies, to generate a report of patients matched to said quadruple-blind clinical studies, to utilize said communications component so as to electronically notify in real time said medical practices associated with said matched patients, and to calculate, utilizing said compensation database component, a fee to be paid to a participant for referral procedures performed in referral of said matched patients to said quadruple-blind clinical study to which said patient is matched.

2. The method according to claim 1, further comprising:
confirming said communications component sends at least one of said reports over said communications link to said medical practices in said medical practice database to electronically notify in real time said medical practices of said matched patients for referral, and wherein said medical practices locate and refer said matched patients to said quadruple-blind clinical study to which each said patient is matched; and
notifying in real time said clinical study entities of referral of said patients to said medical studies over said communications link utilizing said communication component, and said database components are updated utilizing said processor;

3. The method according to claim 1, wherein each of said plurality of unique identifiers corresponds to:
each of said plurality of quadruple-blind clinical studies,
each of said plurality of medical practices,
a medical director,
a board of medical advisers, and
each of said plurality of patient records.

4. The method according to claim 1, further comprising:
listing each of said plurality of quadruple-blind clinical studies, via a listing component, in said quadruple-blind clinical studies database component, wherein said listing component is further configured to list said quadruple-blind clinical study on an internet site, online professional journals, online newspapers, and online magazines that reach a target audience for the quadruple-blind clinical study.

5. The method according to claim 1, further comprising:
searching, via a searching component, said quadruple-blind clinical studies database and said medical practice database, wherein said communications component is adaptable to receive queries from said medical practices and said quadruple-blind clinical studies via said searching component.

6. The method according to claim 1, wherein said compensation database component is further operative to maintain an electronic copy of said binding legal written instrument, and wherein said legally binding written instrument includes a contract, license agreement, personal services and management agreement, and consultation agreement.

7. The method according to claim 5, wherein said compensation component is further operative to transfer monetary payment to a medical researcher, wherein said monetary payment is calculated, via a processor, in conjunction with said compensation database component, at a flat fee per month, and wherein said flat fee per month will reflect the amount of hours spent on each of said plurality of patient records by said medical researcher.

8. The method according to claim 7, wherein said compensation database component is further adapted to compensate said medical researcher pursuant to the requirements as set out by said consultation agreement.

9. The method according to claim 7, wherein said compensation database component is further adapted to compensate a referral physician pursuant to the requirements as set out by said consultation agreement.

10. The method according to claim 7, wherein said verification component is further configured to store an electronic version of said legally binding written instrument.

11. The method according to claim 1, wherein said compensation component is further operative to transfer monetary payment to a medical researcher, wherein said monetary payment is calculated, via the processor, based on the average hourly rate of a medical researcher pursuant to a Medscape Physician Compensation Report.

12. The method according to claim 1, wherein said network includes the internet.

13. The method according to claim 1, wherein said network further utilizes a Cisco 4451-x Integrated Services Router.

14. The method according to claim 1, wherein said system can be implemented in Ireland.

15. The method according to claim 1, wherein said clinical study entities includes medical manufacturing corporations in both Asia and Europe.

16. The method according to claim 1, wherein said medical practices are located within the United States.

17. The method according to claim 1, wherein said clinical study entities includes a pharmaceutical or medical device company with a principal place of business in Asia.

18. The method according to claim 2, wherein said medical director is responsible for inputting data into said compensation component.

19. The method according to claim 8, wherein said legally binding instruments are signed in Ireland.

20. The method according to claim 1, wherein said clinical study entities includes a pharmaceutical or medical device company with a principal place of business in Europe.
